(19) United States
(12) Patent Application Publication
(10) Pub. No.: US 2007/0038029 A1
(43) Pub. Date: Feb. 15, 2007

(54) ENDOSCOPE

(75) Inventor: Noriko OTA, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/462,554

(22) Filed: Aug. 4, 2006

(30) Foreign Application Priority Data
Aug. 9, 2005 (JP) P2005-230540

(51) Int. Cl. A61B 1/06

(52) U.S. Cl. 600/167; 600/168; 600/118

(57) ABSTRACT

An endoscope includes a light source, an objective optical system, an optical system mover, a light amount detector, and a subject image generator. The light source emits illuminating light on a subject, and the reflected light of the illuminating light reflected on the subject, enters an objective optical system. The optical system mover moves the objective optical system in a direction of an optical axis of the objective optical system. The light amount detector detects the amount of the reflected light. The subject image generator generates image signals of the subject based on the reflected light. When an amount of the reflected light entering the objective optical system decreases, the optical system mover moves the objective optical system farther from the subject, and when an amount of the reflected light entering the objective optical system increases, the optical system mover moves the objective optical system closer to the subject, so that the objective optical system is focused on the subject.

### BACKGROUND OF THE INVENTION

1. Field of the Invention

The present invention relates to an endoscope, especially to an endoscope having an auto focus function.

2. Description of the Related Art

An endoscope having an auto focus function using a so-called hill-climbing method based on luminance signals obtained from image signals, is known.

On the other hand, an endoscope that detects the amount of reflected light of illuminating light emitted by a light source reflected on a subject, calculates a subject distance based on an opening position of an aperture for adjusting an intensity of the illuminating light (that is, based on an amount of reflected light) and adjusts the focal distance is also known.

In an auto focusing by the hill-climbing method, it sometimes takes a relatively long time to focus. Especially, in an observation optical system for endoscopes, because a subject distance is short and an optical system is frequently moved ordinarily, it takes a long time to focus using the hill-climbing method, so that achieving immediate response for focusing consistently is difficult.

### SUMMARY OF THE INVENTION

Therefore, an objective of the present invention is to provide an endoscope that has an auto focus function for adjusting focal distance immediately and reliably.

A focusing device for an endoscope, according to the present invention, includes a light source, an objective optical system, an optical system mover, and a light amount detector. The light source emits illuminating light on a subject, and the reflected light of the illuminating light reflected on the subject enters into the objective optical system. The optical system mover moves the objective optical system in a direction of an optical axis of the objective optical system. The light amount detector detects the amount of the reflected light. When an amount of the reflected light entering into the objective optical system decreases, the optical system mover moves the objective optical system farther from the subject, and when an amount of the reflected light entering into the objective optical system increases, the optical system mover moves the objective optical system closer to the subject, so that the objective optical system is focused on the subject.

An endoscope, according to the present invention, includes a light source, an objective optical system, an optical system mover, a light amount detector, and a subject image generator. The light source emits illuminating light on a subject, and the reflected light of the illuminating light reflected on the subject, enters into an objective optical system. The optical system mover moves the objective optical system in a direction of an optical axis of the objective optical system. The light amount detector detects the amount of the reflected light. The subject image generator generates image signals of the subject based on the reflected light. When an amount of the reflected light entering into the objective optical system decreases, the optical system mover moves the objective optical system farther from the subject, and when an amount of the reflected light entering into the objective optical system increases, the optical system mover moves the objective optical system closer to the subject, so that the objective optical system is focused on the subject.

An auto focusing device for an endoscope, according to the present invention, includes a viewing optical system, a light amount detector, and a focusing controller. The viewing optical system has a focusing optical system which is movable for focusing. The light amount detector detects amount of light enters the viewing optical system from a subject. The focusing controller controls the focusing optical system based on signals from the light amount detector so that an optical image of the subject is focused on a predetermined surface. The focusing controller determines the moving direction of the focusing optical system at the starting time of controlling the focusing optical system, depending on whether the amount of light detected by the light amount detector is increased or decreased.

### BRIEF DESCRIPTION OF THE DRAWINGS

The present invention will be better understood from the description of the preferred embodiment of the invention set forth below together with the accompanying drawings, in which.

FIG. 1 is a block diagram of an endoscope of the embodiment;

FIG. 2 is a side sectional view representing an end of a video scope when a moving lens is closer to the far end side than to the focused position;

FIG. 3 is a side sectional view representing an end of the video scope when the moving lens is in the focused position;

FIG. 4 is a side sectional view representing the end of the video scope when the moving lens is closer to near end side than to the focused position;

FIG. 5 is a view representing a relation between a strength and frequency of image signals output from a CCD when an objective optical system is not focused;

FIG. 6 is a view representing the relation between the strength and frequency of image signals output from the CCD when an objective optical system is focused;

FIG. 7 is a view representing a relation between a distance from the CCD to the moving lens and an evaluation value, that is a strength of the signals at a predetermined frequency;

FIG. 8 is a view representing the end of the video scope that is moving by varying the distance from a subject;

FIG. 9 is a view representing a change of brightness of a subject image as the end of the video scope is approaches a subject;

FIG. 10 is a view representing a change of brightness of a subject image as the end of the video scope departs from a subject, and

FIG. 11 is a flowchart of a focus control routine representing a focus control in an endoscope.

### DESCRIPTION OF THE PREFERRED EMBODIMENT

Hereinafter, the preferred embodiment of the present invention is described with reference to the attached drawings.

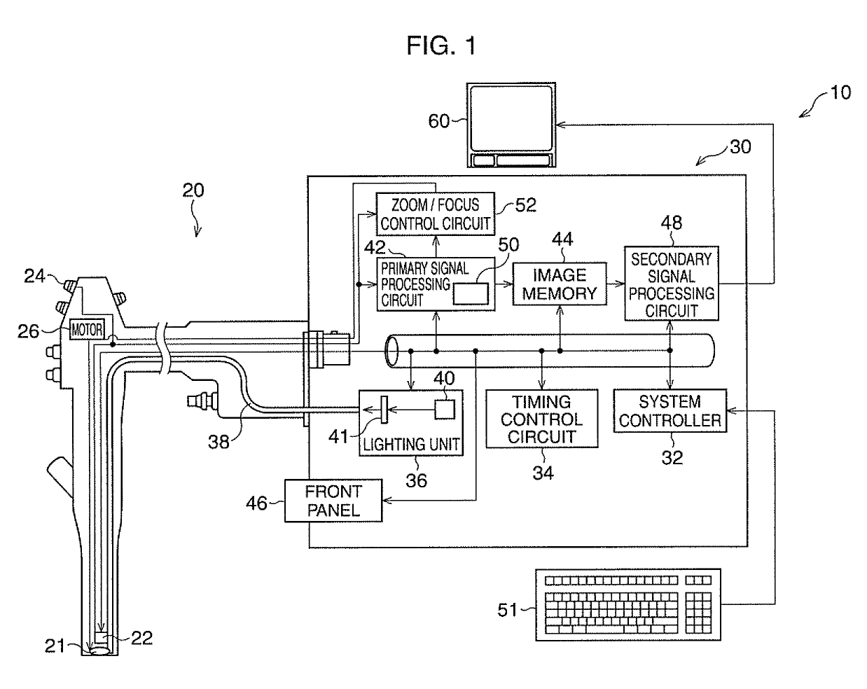

As shown in FIG. 1, an endoscope 10 includes a video scope 20 and a processor 30. The video scope 20 is used for photographing inside a body cavity. The processor 30 processes image signals transferred from the video scope 20. To the processor 30, a keyboard 50 for inputting order signals and other information, and a monitor 60 for displaying a subject image, are connected.

In the processor 30, a system controller 32 for controlling the entirety of the processor 30, a timing control circuit 34 for controlling signal processing timing in other circuits, a lighting unit 36, and other components are provided. A light source 40 in the lighting unit 36 emits illuminating light under the control of the system controller 32. The illuminating light enters a light guide 38 after its amount is adjusted by an aperture 41. The illuminating light passes through the light guide 38, and is emitted on a body cavity from the end of the video scope 20.

The reflected light of the illuminating light reflected on a subject enters an objective lens system 21 at the end of the video scope 20. The objective lens system 21 is represented as a simple lens in FIG. 1, although a plurality of lenses are included in the objective lens system 21 in practice. The reflected light passing through the objective lens system 21 reaches a light-receiving surface of the CCD 22. Then, image signals representing a subject are generated by the CCD 22. Further, luminance signals and color-difference signals are generated by processing the image signals. The luminance signals and color-difference signals are transferred to a primary signal processing circuit 42, and are stored in an image memory 44 after further processes are carried out in the primary signal processing circuit 42.

Image data, including the luminance signals and color-difference signals, are output from the image memory 44 to the monitor 60 via a secondary signal processing circuit 48. As a result, a real-time moving image of a subject is displayed on the monitor 60.

A freeze button (not shown) is provided on the video scope 20. When the freeze button is depressed, signals for generating a still image are transferred to the system controller 32, and image data of a still image are generated. Generated image data of a still image are stored in the image memory 44, and transferred to the secondary signal processing circuit 48. In the secondary signal processing circuit 48, predetermined processes are carried out on the image data, and the image data are transferred to the monitor 60. As a result, a still image is displayed on the monitor 60.

The endoscope 10 has a zoom function for controlling the focal distance of the objective lens system 21, and a auto focus function for focusing on a subject. On the video scope 20, a zoom focus button 24 is provided. When the zoom focus button 24 is depressed, the magnification of an image varies according to the operation of the zoom focus button 24, and a moving lens included in the objective lens system 21 is moved in a direction of the optical axis of the objective lens system 21, to a focused position to be focused on a subject using the so-called hill-climbing method, as explained below.

That is, when signals representing that the zoom focus button 24 is depressed are transferred to the system controller 32, a zoom/focus control circuit 52 causes the moving lens of the objective lens system 21 to move to the focused position to be focused on a subject, and further causes the moving lens to move to adjust the focal distance to correspond to the predetermined image magnification by controlling a motor 26, based on a command from the system controller 32.

In the primary signal processing circuit 42, a light-amount detecting unit 50 for detecting the amount of the reflected light of the illuminating light entering the CCD 22 off a subject via the objective lens system 21 is provided. In the light-amount detecting unit 50, signals representing the amount of the reflected light (called “AE signals” hereinafter) are generated based on the luminance signals, and the AE signals are transferred to the system controller 32, for the exposure control.

The system controller 32 adjusts an aperture value of the aperture 41 and the shutter speed of the electronic shutter of the CCD 22, based on the received AE signals and the sensitivity of the CCD 22. At this time, from the system controller 32, command signals for commanding opening or closing of the aperture 41 to the predetermined aperture position are transferred to the lighting unit 36, and other command signals for commanding the shutter speed are transferred to the CCD 22, respectively.

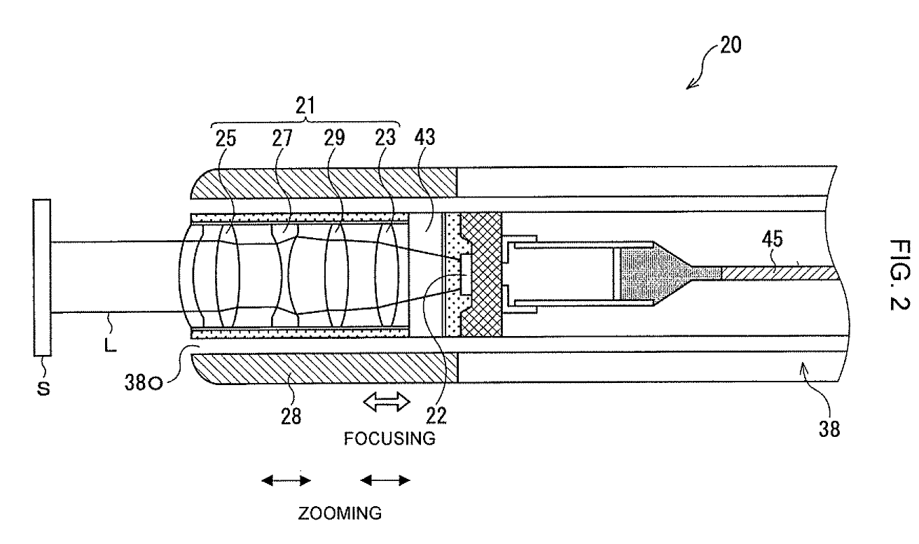

In the endoscope 10, focusing control of the objective lens system 21 is carried out by the so-called hill climbing method, as explained below. First, as shown in FIGS. 2 to 4, the illuminating light is emitted on a subject S from an emitting end surface 380 of the light guide 38, which is inside an end part 28 of the video scope 20. While the reflected light L of the illuminating light enters the objective lens system 21, including the first moving lens 23 for focusing and varying focal distance, the second moving lens 27 for varying focal distance, and the first and the second non-moving lenses 25 and 29. The first moving lens 23 is moved in the direction of the optical axis of the objective lens system 21 for focusing, The second moving lens 27 is also moved in the direction of the optical axis of the objective lens system 21 with the first moving lens 23, so that the zooming is carried out by changing relative position of the first and second moving lenses 23 and 27.

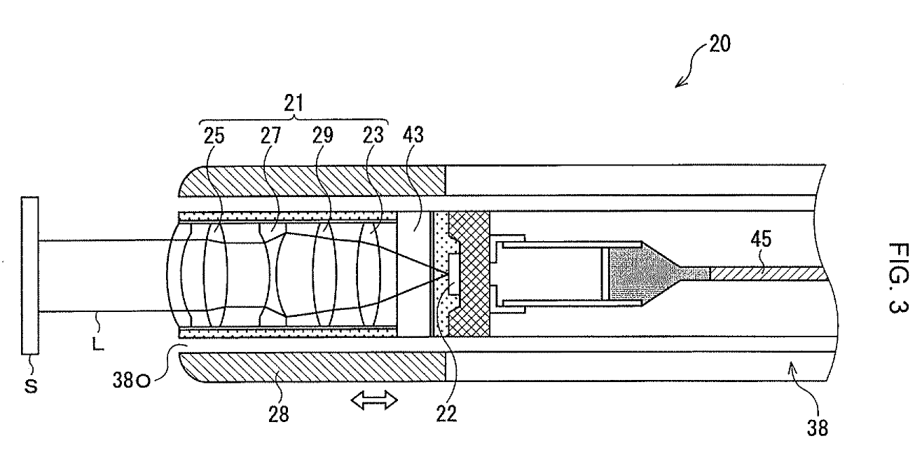

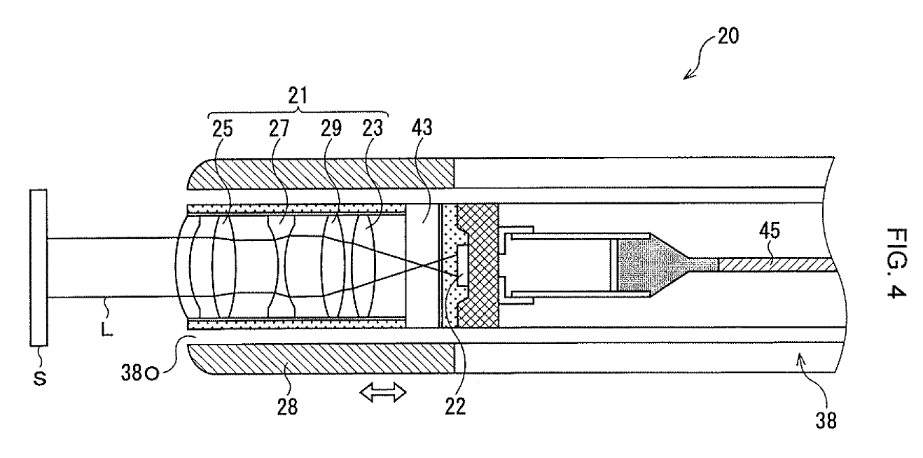

The first moving lens 23 is moved, for example, between a lens position which is in the far end side and close to the CCD 22, shown in FIG. 2, and another lens position which is in the near end side and distant from the CCD 22, as shown in FIG. 4, via the focused position shown in FIG. 3. The reflected light L passes through the objective lens system 21, and enters the CCD 22 via a cover glass 43. Image signals based on the reflected light L are transferred to the processor 30 via a signal cable 45.

In the system controller 32 (see FIG. 1), the strength of the image signals output from the CCD 22 is calculated for each frequency, based on the image signals transferred from the CCD 22 to the primary signal processing circuit 42. The strength of the image signals at a relatively high frequency is ordinarily stronger when the objective lens system 21 is focused on the subject S; that is, when the first moving lens 23 is in the focused position. On the contrary, the strength of the image signals at a relatively high frequency is ordinarily weaker when the objective lens system 21 is not focused on the subject S. Further, the distance between the actual lens position of the first moving lens 23 and the focused position becomes larger as the strength of the image signals at a relatively high frequency becomes weaker.

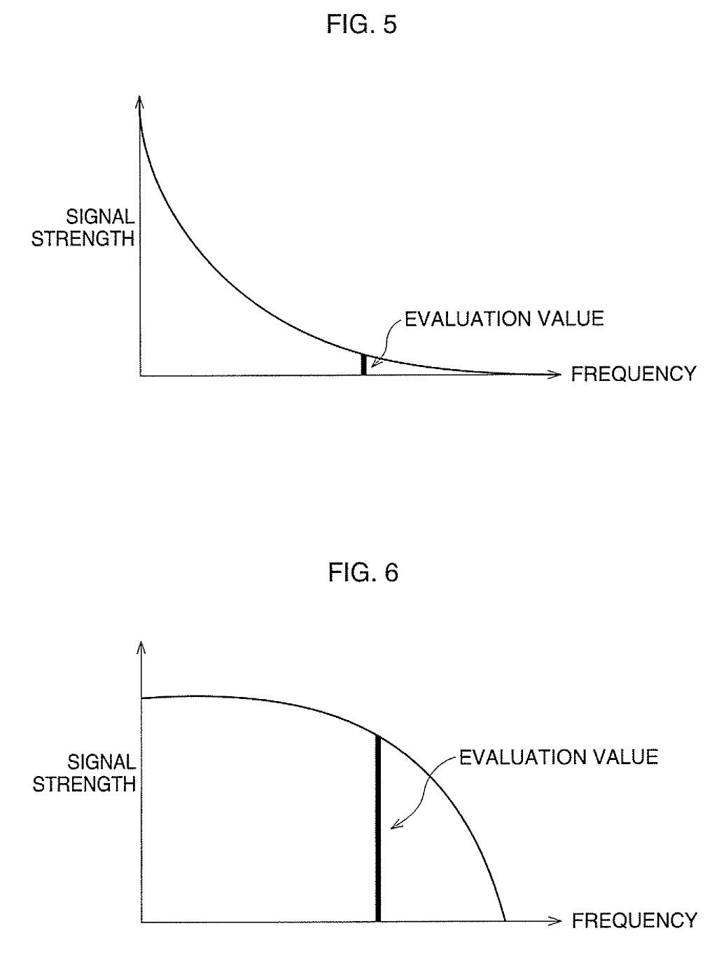

Therefore, when the objective lens system 21 is not focused (see FIGS. 2 and 4), the relation between the strength and the frequency of the image signals is exemplified as shown in FIG. 5, where the strength of the image signals at the higher frequency is small. On the other hand, when the objective lens system 21 is focused (see FIG. 3), the strength of the image signals at the higher frequency is large, as exemplified in FIG. 6. In the system controller 32, the image signal strength at a predetermined relatively high frequency is detected as an evaluation value.

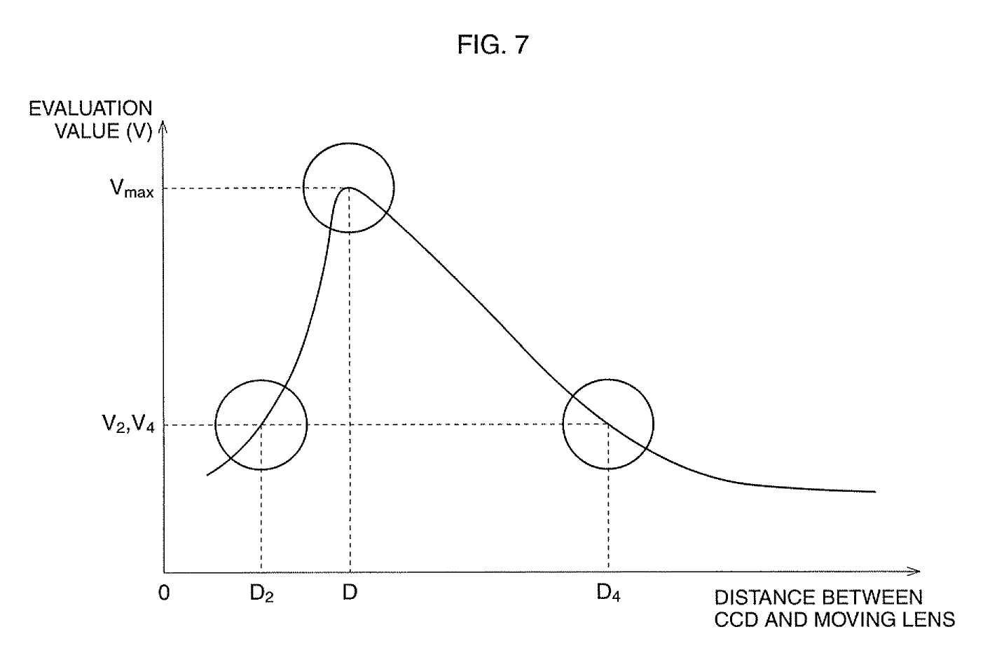

Distribution data of the relation between the evaluation value and the distance between the first moving lens 23 and the CCD 22, as exemplified in FIG. 7, is obtained by calculating the evaluation values at various lens positions of the moved first moving lens 23. The system controller 32 determines that the lens position corresponding to the peak of the evaluation values is the focused position. Then, the zoom/focus control circuit 52 controls the motor 26, so that the first moving lens 23 is moved to the focused position, and the objective lens system 21 is focused on the subject S.

For example, in the distribution data of the evaluation value shown in FIG. 7, the maximum evaluation value “Vₘₐₓ” at the lens position in FIG. 3, where the distance between the first moving lens 23 and the CCD 22, which is the distance “D” is greater than the evaluation values “V₂” and “V₄” at the lens positions in FIGS. 2 and 4, where the distances between the first moving lens 23 and the CCD 22 are the distances “D₂” and “D₄” respectively. Therefore, in this case, the position of the first moving lens 23 represented in FIG. 3 is determined to be the focused position. Then, the first moving lens 23 is moved to the focused position, and the focusing operation ends. Note that the data of the evaluation value is stored in a data memory (not shown) until the focused position is detected.

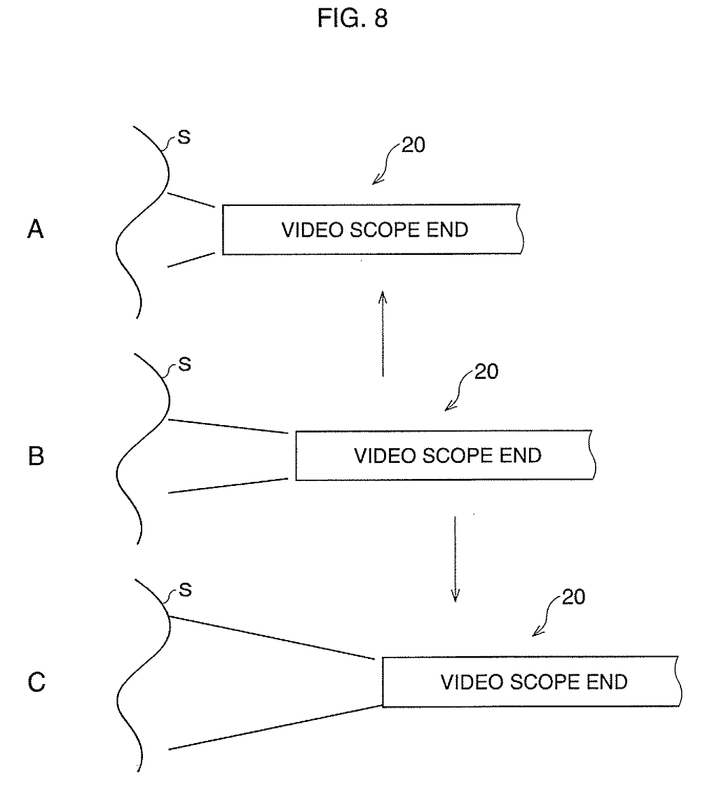

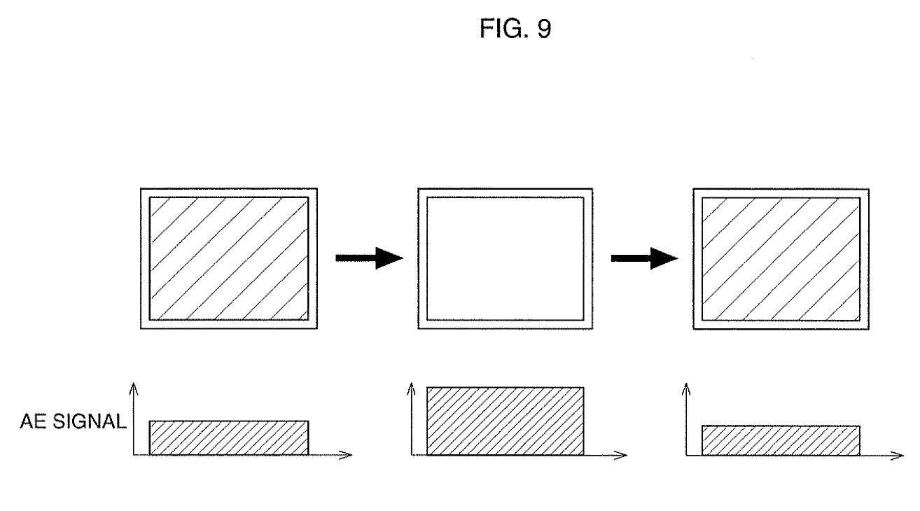

As shown in FIG. 8, when the end of the video scope 20 approaches the subject S from the position represented by B to the position represented by A under a constant amount of the illuminating light, the amount of the reflected light L represented by the AE signals increases. Therefore, the aperture value of the aperture 41 (see FIG. 1) increases and the aperture 41 becomes more closed immediately, under the control of the system controller 32. As a result, the amount of the reflected light L returns to the amount represented by the AE signals before the end of the video scope 20 had been moved (see FIG. 9).

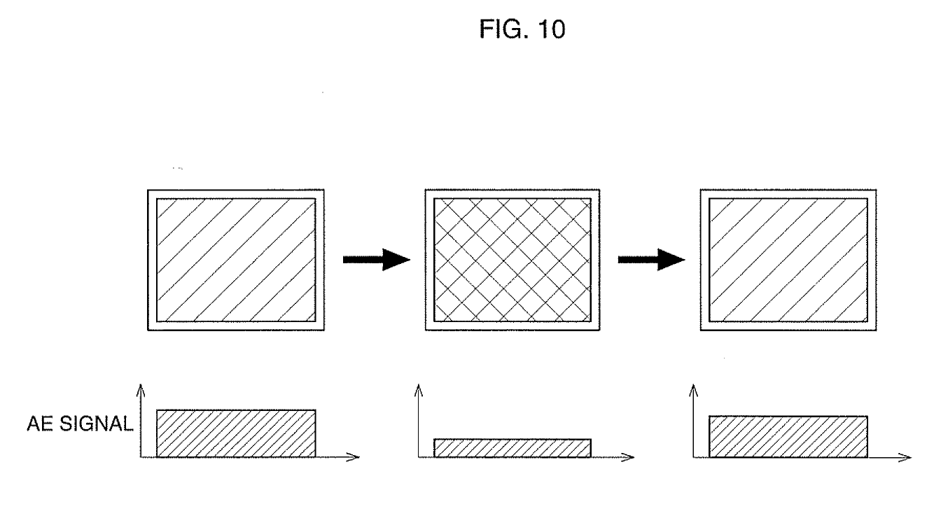

On the other hand, when the end of the video scope 20 is moved farther from the subject S, from the position represented by B to the position represented by C under a constant amount of an illuminating light, the amount of the reflected light L represented by the AE signals decreases. In this case, the aperture value of the aperture 41 is decreased under the control of the system controller 32. Then, the amount of the reflected light L returns to the amount represented by the AE signals before the end of the video scope 20 had been moved (see FIG. 10).

As explained above, when the amount of the reflected light L varies under a constant amount of the illuminating light, the distance between the end of the video scope 20, including the CCD 22 and the subject S also varies, therefore, focusing is required. In the focusing operation by the so-called hill climbing method, first, the first moving lens 23 is moved closer to either the far end or the near end, depending on whether the amount of the reflected light L is increasing or decreasing.

That is, if the amount of the reflected light L increases when the amount of the illuminating light is constant, it is determined that the distance between the video scope 20 and the subject S has become shorter. Therefore, the system controller 32 (see FIG. 1) controls the zoom/focus control circuit 52 so that the first moving lens 23 is moved, and the distance from the CCD 22 to the first moving lens 23 becomes longer than that to the first moving lens 23 just before the amount of the reflected light L had increased. That is, the first moving lens 23 is moved to the near end side (i.e., closer to the subject S and farther from the CCD 22). Next, the so-called hill-climbing method is carried out. The system controller 32 also controls aperture 41 for exposure control when the amount of the reflected light L increases.

On the other hand, if the amount of the reflected light L decreases when the amount of the illuminating light is constant, it is determined that the distance between the video scope 20 and the subject S has become longer. Therefore, the system controller 32 controls the zoom/focus control circuit 52 so that the first moving lens 23 is moved, and the distance from the CCD 22 to the first moving lens 23 becomes shorter than that to the first moving lens 23 just before the amount of the reflected light L had decreased. That is, the first moving lens 23 is moved to the far end side (i.e., farther from the subject S and closer to the CCD 22). Next, the so-called hill-climbing method is carried out.

As explained above, in the auto focusing operation by the hill-climbing method, the first moving lens 23 is moved in a suitable direction, so that the required moving distance of the first moving lens 23 and the required time for focusing are both less than those in the case where the direction of the first movement of the first moving lens 23 is not determined.

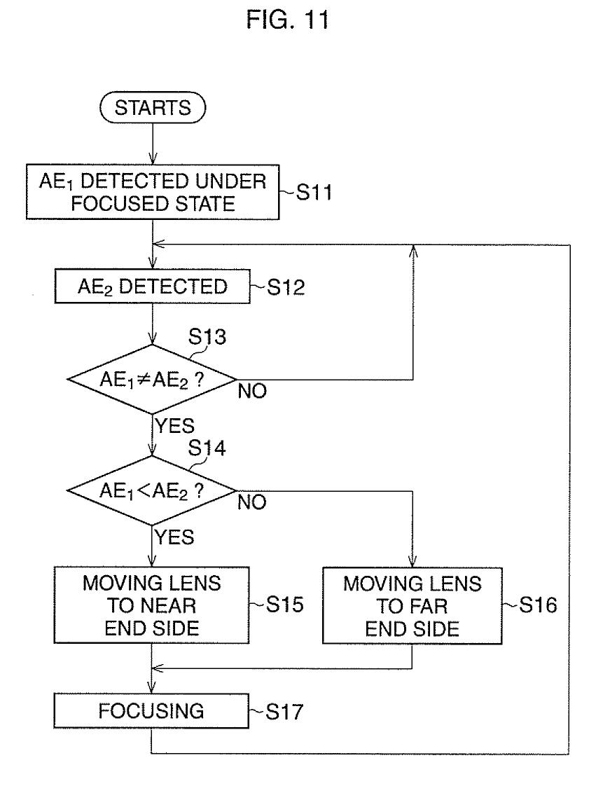

The focus control routine (see FIG. 11) starts when the illuminating light is emitted by the light source 40 (see FIG. 1). At step S11, the “AE₁” signal representing the amount of the reflected light L is detected by the system controller 32 under the objective lens system 21 focused on the subject S, and the process proceeds to step S12. At step S12, the “AE₂” signal is newly detected by the system controller 32 after the predetermined time period elapses, and the process proceeds to step S13. At step S13, it is determined whether the values of the “AE₁” signal and the “AE₂” signal are equal or not; that is, whether the amounts of the reflected light L represented by the “AE₁” signal and the “AE₂” signal are equal or not. If it is determined that the amounts of the reflected light are equal, the process returns to step S12, and if it is determined that the amounts of the reflected light are not equal, the process proceeds to step S14.

At step S14, whether the amount of the reflected light L represented by the “AE₁” signal is smaller than that represented by the “AE₂” signal or not is determined. When it is determined that the amount of the reflected light L represented by the “AE₁” signal is smaller than that represented by the “AE₂” signal, the process proceeds to step S15, and when it is determined that the amount of the reflected light L represented by the “AE₁” signal is larger than that represented by the “AE₂” signal, the process proceeds to step S16.

At step S15, the first moving lens 23 is moved to the near end side, and the process proceeds to step S17. At step S16, the first moving lens 23 is moved to the far end side, and the process proceeds to step S17. At step S17, the first moving lens 23 is moved to the focused position by the hill-climbing method, and the process returns to step S11.

As explained above, in this embodiment, the objective lens system 21 is immediately focused by detecting the amount of the reflected light under the condition of no light other than the illuminating light emitted by the light source 40, and by moving the first moving lens 23 in a suitable direction closer to the focused position by an auto focus operation. Further, the objective lens system 21 can be accurately focused, because the first moving lens 23 is moved in a suitable direction first, before carrying out the hill-climbing method.

The objective lens system 21 is not limited to a zoom lens, and the numbers and arrangements of the first moving lens 23, and the first to third non-moving lenses 25, 27, and 29, are not limited to those in the embodiment. Further, instead of the zoom focus button 24, independent switches for each of the zoom function and the auto focus function may be provided.

The auto focus method is not limited to the contrast method in the embodiment where the high frequency component in the image signals is detected.

If the first moving lens 23 being moved in the focusing operation and the moving lens being moved in a zooming operation are different in the objective lens system 21, a plurality of motors 26 may be provided for moving each of the different moving lenses. Further, the position of the motor 26 is not limited to that in the embodiment, for example, the motor 26 may be arranged in the end of the video scope 20.

The invention is not limited to that described in the preferred embodiment; namely, various improvements and changes may be made to the present invention without departing from the spirit and scope thereof.

The present disclosure relates to subject matter contained in Japanese Patent Application No. 2005-230540 (filed on Aug. 9, 2005) which is expressly incorporated herein, by reference, in its entirety.

1. A focusing device for an endoscope, said focusing device comprising;
a light source that emits illuminating light on a subject;
an objective optical system to which reflected light of said illuminating light reflected on said subject light enters;
an optical system mover that moves said objective optical system in a direction of an optical axis of said objective optical system; and
a light amount detector that detects amount of said reflected light;
wherein when an amount of said reflected light entering said objective optical system decreases, said optical system mover moves said objective optical system farther from said subject, and when an amount of said reflected light entering said objective optical system increases, said optical system mover moves said objective optical system closer to said subject, so that said objective optical system is focused on said subject.

2. The focusing device according to claim 1, further comprising a focus judge that determines whether said objective optical system is focused on said subject or not.

3. The focusing device according to claim 1, further comprising a zoom controller that controls focal distance of said objective optical system.

4. The focusing device according to claim 3, further comprising a switch for said optical system mover that moves said objective optical system, wherein when said switch is turned on, said optical system mover moves said objective optical system, and said zoom controller starts.

5. The focusing device according to claim 4, wherein when said switch is turned on, said optical system mover moves said objective optical system to a focused position.

6. An endoscope comprising;
a light source that emits illuminating light on a subject;
an objective optical system to which reflected light of said illuminating light reflected on said subject light enters;
an optical system mover that moves said objective optical system in a direction of an optical axis of said objective optical system;
a light amount detector that detects amount of said reflected light; and
a subject image generator that generates image signals of said subject based on said reflected light;
wherein when an amount of said reflected light entering said objective optical system decreases, said optical system mover moves said objective optical system farther from said subject, and when an amount of said reflected light entering said objective optical system increases, said optical system mover moves said objective optical system closer to said subject, so that said objective optical system is focused on said subject.

7. The endoscope according to claim 6, further comprising an exposure adjuster that adjusts an exposure for generating said image signals, based on an amount of said reflected light.

8. The endoscope according to claim 7, wherein said exposure adjuster comprises an aperture that adjusts an amount of said illuminating light.

9. The endoscope according to claim 6, further comprising a focus judge that determines whether said objective optical system is focused on said subject or not, wherein said focus judge determines that said objective optical system is focused on said subject when a strength of said image signals is maximum.

10. The endoscope according to claim 9, wherein said focus judge determines that said objective optical system is focused on said subject when a strength of said image signals is maximum at a predetermined frequency.

11. An auto focusing device for an endoscope, said auto focusing device comprising;
a viewing optical system that has a focusing optical system which is movable for focusing;
a light amount detector that detects amount of light enters said viewing optical system from a subject; and
a focusing controller that controls said focusing optical system based on signals from said light amount detector so that an optical image of said subject is focused on a predetermined surface;
wherein said focusing controller determines the moving direction of said focusing optical system at the starting time of controlling said focusing optical system, depending on whether said amount of light detected by said light amount detector is increased or decreased.

12. The auto focusing device according to claim it, wherein said focusing controller determines said moving direction for a subject closer to said auto focusing device than the current subject to be focused, when said amount of light is increased, and said focusing controller determines said moving direction for a subject farther from said auto focusing device than the current subject to be focused, when said amount of light is decreased.

13. The auto focusing device according to claim 12, wherein said focusing controller moves said focusing optical system to be closer to said subject, when said amount of light is increased, and said focusing controller moves said focusing optical system to be closer to said predetermined surface, when said amount of light is decreased.

14. The auto focusing device according to claim 13, wherein said light amount detector is a CCD.

15. The auto focusing device according to claim 11, wherein said light amount detector is arranged on said predetermined surface.
